(19) United States
(12) Patent Application Publication
(10) Pub. No.: US 2021/0380935 A1
(43) Pub. Date: Dec. 9, 2021

(54) Cell Culture Surface Compositions, Systems, and Methods

(71) Applicant: Georgia Tech Research Corporation, Atlanta, GA (US)

(72) Inventors: Molly E. Ogle, Atlanta, GA (US); Gilad Doron, Atlanta, GA (US); Johnna S. Temenoff, Atlanta, GA (US)

(21) Appl. No.: 17/283,383

(22) PCT Filed: Oct. 8, 2019
(86) PCT No.: PCT/US2019/055165

§ 371 Date: Apr. 7, 2021

Related U.S. Application Data
(60) Provisional application No. 62/742,555, filed on Oct. 8, 2018.

(51) Int. Cl. C12N 5/00; C12N 5/0775

(52) U.S. Cl. CPC C12N 5/0068; C12N 5/0663; C12N 2533/30; C12N 2533/50; C12N 2533/20; C12N 2537/10; C12N 2533/70

(57) ABSTRACT

Disclosed herein is a cell culture surface, comprising: a polymer material comprising an acrylate functional group; and one or more functional compounds comprising one or more functional moieties, at least one moiety from the one or more functional moieties configured to adhere to animal cells; wherein the cell culture surface has a compressive modulus between 10 kPa to 1000 kPa. Also disclosed herein are systems and methods for making and using the same.

### CROSS-REFERENCE TO RELATED APPLICATIONS

This application claims the benefit of U.S. Provisional Patent Application No. 62/742,555, filed 8 Oct. 2018, the entire contents and substance of which is incorporated herein by reference in its entirety as if fully set forth below.

### STATEMENT OF RIGHTS UNDER FEDERALLY SPONSORED RESEARCH

This invention was made with government support under Grant No. 1648035 awarded by the National Science Foundation. The government has certain rights in the invention.

### FIELD OF THE DISCLOSURE

The present disclosure relates generally to cell culture surface compositions. Particularly, embodiments of the present disclosure relate to cell culture surface compositions for culturing cells, and systems and methods of making and using the same.

### BACKGROUND

Mesenchymal stromal cells (MSCs) are highly paracrine active cells which are of great clinical interest due to their immunomodulatory and pro-regenerative properties when transplanted in vivo. The therapeutic capacity of MSCs is primarily attributed to their release of immunomodulatory, angiogenic, and trophic factors which are collectively known as the secretome. Pro-regenerative secretory activity of MSCs in vitro is correlated with in vivo activity, suggesting that the in vitro secretome can be related to MSC therapeutic function in some applications. To achieve therapeutic cell numbers in the range of millions of cells per kg patient body weight from bone marrow, adipose, umbilical cord primary cells, and the like, most clinical therapy protocols employ several rounds of MSC expansion. However, these standard culture expansion protocols progressively decrease MSC secretory potency over population doublings, leading to significant changes in MSC therapeutic function in vivo. In addition, MSCs undergo a progressive increase in senescence and decrease in expansion during standard in vitro culture which restricts the number of cells produced from a single source and alters their gene expression, differentiation, and secretory phenotypes. These issues establish a conundrum by which the primary cell source is limited and must be expanded for clinical scale, but expansion leads to reduced therapeutic potency and is further restricted by senescence. Furthermore, many serum additives are used to enhance current cell culture methods. Such serum additives or other growth stimulating factors are commonly xenogenic sources with variable composition and represent a high contribution to cost of cell cultures. Current cell culture surfaces used for the expansion and production of MSCs are, therefore, not optimized for the cost-effective production of highly-secretory and non-senescent cells.

What is needed, therefore, is a cell culture surface for culturing animal cells, such as MSCs, that can present increased proliferation, decreased senescence, enhanced secretory activity, and reduce the concentration of additional serum needed for the cell culture. Embodiments of the present disclosure address this need as well as other needs that will become apparent upon reading the description below in conjunction with the drawings.

### BRIEF SUMMARY OF THE INVENTION

The present disclosure relates generally to cell culture surface compositions.

Particularly, embodiments of the present disclosure relate to cell culture surface compositions for culturing cells, and systems and methods of making and using the same. An exemplary embodiment of the present invention can provide a cell culture surface, comprising: a polymer material comprising an acrylate functional group; and one or more functional compounds comprising one or more functional moieties, at least one moiety from the one or more functional moieties configured to adhere to animal cells; wherein the cell culture surface has a compressive modulus between 10 kPa to 1000 kPa.

In any of the embodiments disclosed herein, the one or more functional compounds can comprise at least one glycosaminoglycan (GAG) compound.

In any of the embodiments disclosed herein, the at least one GAG compound can be selected from the group consisting of: heparins, desulfated heparins, hyaluronic acids, and linear repeating disaccharides.

In any of the embodiments disclosed herein, the one or more functional compounds can comprise at least one cadherin-engaging compound.

In any of the embodiments disclosed herein, the one or more functional compounds can comprise at least one integrin-engaging compound.

In any of the embodiments disclosed herein, the one or more functional compounds can comprise at least one carbohydrate compound.

In any of the embodiments disclosed herein, the polymer material can comprise poly(ethylene glycol) diacrylate (PEG-DA).

In any of the embodiments disclosed herein, the polymer material can have a molecular weight of from 600 Da to 10000 Da.

In any of the embodiments disclosed herein, the polymer material can be present in the cell culture surface in an amount of from 1% to 75% by weight per unit volume.

In any of the embodiments disclosed herein, the cell culture surface can further comprise an initial quantity of animal cells distributed on a surface of the cell culture surface.

In any of the embodiments disclosed herein, the initial quantity of animal cells can increase by a factor of from 1 to 10 after 4 days of culturing.

Another embodiment of the present disclosure can provide a method of culturing cells, comprising: distributing a culture of animal cells on a cell culture surface having a compressive modulus from 10 kPa to 1000 kPa, the cell culture surface comprising: a polymer material comprising an acrylate functional group; and one or more functional compounds comprising one or more functional moieties, at least one moiety from the one or more functional moieties configured to adhere to animal cells; incubating the culture of animal cells such that the culture of animal cells attaches to the cell culture surface; and recovering the culture of animal cells from the cell culture surface.

In any of the embodiments disclosed herein, the culture of animal cells can comprise mesenchymal stem cells.

In any of the embodiments disclosed herein, the one or more functional compounds can comprise at least one glycosaminoglycan (GAG) compound.

In any of the embodiments disclosed herein, the at least one GAG compound can be selected from the group consisting of: heparins, desulfated heparins, hyaluronic acids, and linear repeating disaccharides.

In any of the embodiments disclosed herein, the one or more functional compounds can comprise at least one cadherin-engaging compound.

In any of the embodiments disclosed herein, the one or more functional compounds can comprise at least one integrin-engaging compound.

In any of the embodiments disclosed herein, the one or more functional compounds can comprise at least one carbohydrate compound.

In any of the embodiments disclosed herein, the polymer material can comprise poly(ethylene glycol) diacrylate (PEG-DA).

In any of the embodiments disclosed herein, the polymer material can have a molecular weight of from 600 Da to 10000 Da.

In any of the embodiments disclosed herein, the polymer material can be present in the cell culture surface in an amount of from 1% to 75% by weight per unit volume.

In any of the embodiments disclosed herein, the culture of animal cells can increase by a factor of from 1 to 10 after 4 days of culturing.

Another embodiment of the present disclosure can provide a method of making a cell culture surface, the method comprising: dissolving a polymer material comprising an acrylate functional group in a first solvent to create a first solution; functionalizing the first solution with one or more functional compounds comprising one or more functional moieties, at least one moiety from the one or more functional moieties configured to adhere to animal cells; and cross-linking the first solution to obtain a cell culture surface, the cell culture surface having a compressive modulus of from 10 kPa to 1000 kPa.

In any of the embodiments disclosed herein, the one or more functional compounds can comprise at least one glycosaminoglycan (GAG) compound.

In any of the embodiments disclosed herein, the at least one GAG compound can be selected from the group consisting of: heparins, desulfated heparins, hyaluronic acids, and linear repeating disaccharides.

In any of the embodiments disclosed herein, the one or more functional compounds can comprise at least one cadherin-engaging compound.

In any of the embodiments disclosed herein, the one or more functional compounds can comprise at least one integrin-engaging compound.

In any of the embodiments disclosed herein, the one or more functional compounds can comprise at least one carbohydrate compound.

In any of the embodiments disclosed herein, the polymer material can comprise poly(ethylene glycol) diacrylate (PEG-DA).

In any of the embodiments disclosed herein, the polymer material can have a molecular weight of from 600 Da to 10000 Da.

In any of the embodiments disclosed herein, the polymer material can be present in the first solution in an amount of from 1% to 75% by weight per unit volume of the first solution.

These and other aspects of the present invention are described in the Detailed Description of the Invention below and the accompanying figures. Other aspects and features of embodiments of the present invention will become apparent to those of ordinary skill in the art upon reviewing the following description of specific, exemplary embodiments of the present invention in concert with the figures. While features of the present invention may be discussed relative to certain embodiments and figures, all embodiments of the present invention can include one or more of the features discussed herein. Further, while one or more embodiments may be discussed as having certain advantageous features, one or more of such features may also be used with the various embodiments of the invention discussed herein. In similar fashion, while exemplary embodiments may be discussed below as device, system, or method embodiments, it is to be understood that such exemplary embodiments can be implemented in various devices, systems, and methods of the present invention.

### BRIEF DESCRIPTION OF THE DRAWINGS

The accompanying drawings, which are incorporated in and constitute a part of this specification, illustrate multiple embodiments of the presently disclosed subject matter and serve to explain the principles of the presently disclosed subject matter. The drawings are not intended to limit the scope of the presently disclosed subject matter in any manner.

FIG. 1 is a flowchart illustrating a method for culturing cells according to some embodiments of the present disclosure.

FIG. 2 is a flowchart illustrating a method for making a cell culture surface according to some embodiments of the present disclosure.

FIG. 3a illustrates non-limiting examples of one or more functional compounds used in a cell culture surface according to some embodiments of the present disclosure.

FIG. 3b illustrates two non-limiting examples of compositions of cell culture surfaces according to some embodiments of the present disclosure.

FIG. 3c illustrates the compressive modulus for two non-limiting examples of compositions of cell culture surfaces according to some embodiments of the present disclosure.

FIG. 4a illustrates the swelling property of a cell culture surface with various functional compounds according to some embodiments of the present disclosure.

FIG. 4b illustrates the swelling property of a cell culture surface with various functional compounds according to some embodiments of the present disclosure.

FIG. 5a illustrates the fold change in cells cultured on a cell culture surface according to some embodiments of the present disclosure compared to traditional cell culture methods.

FIG. 5b illustrates the fold change in cells cultured on a cell culture surface according to some embodiments of the present disclosure compared to traditional cell culture methods.

FIG. 6a illustrates the quantity of secretome produced by cells cultured on a cell culture surface according to some embodiments of the present disclosure.

FIG. 6b illustrates the quantity of secretome produced by cells cultured on a cell culture surface according to some embodiments of the present disclosure.

FIG. 7a illustrates the HUVEC network length of cells cultured on a cell culture surface according to some embodiments of the present disclosure.

FIG. 7b illustrates the HUVEC network length of cells cultured on a cell culture surface according to some embodiments of the present disclosure.

FIG. 8a illustrates the migration of cells cultured on a cell culture surface according to some embodiments of the present disclosure.

FIG. 8b illustrates the migration of cells cultured on a cell culture surface according to some embodiments of the present disclosure.

FIG. 9a illustrates a process for culturing cells according to some embodiments of the present disclosure.

FIG. 9b illustrates the senescence of cells cultured on a cell culture surface according to some embodiments of the present disclosure compared to traditional cell culture methods.

FIG. 9c illustrates the secretome produced by cells cultured on a cell culture surface according to some embodiments of the present disclosure compared to traditional cell culture methods.

### DETAILED DESCRIPTION

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

As described above, a major problem with current cell culture surfaces and culturing methods for cells, such as mesenchymal stromal cells (MSCs) is that, to achieve therapeutic cell numbers in the range of millions of cells per kg patient body weight from bone marrow, adipose, or umbilical cord primary cells, most clinical therapy protocols employ several rounds of MSC expansion. These standard culture expansion protocols progressively decrease MSC secretory potency over population doublings, leading to significant changes in MSC therapeutic function in vivo. In addition, MSCs undergo a progressive increase in senescence and decrease in expansion during standard in vitro culture which restricts the number of cells produced from a single source and alters their gene expression, differentiation, and secretory phenotypes. These issues establish a conundrum by which the primary cell source is limited and must be expanded for clinical scale, but expansion leads to reduced therapeutic potency and is further restricted by senescence. The embodiments described herein, in contrast, can provide a modular cell culture surface-based culture system to explore the role of combinations of different mechanical and biochemical cues in governing both MSC secretory potency and longevity in culture to improve the clinical-scale manufacture of highly therapeutic MSCs.

To assess culture parameters that may enhance the in vitro production of therapeutic MSCs, the mechanical and biochemical properties consistent with native in vivo perivascular environment of MSCs were considered where they can be highly secretory and capable of long-term self-renewal. One difference between the in vivo MSCs perivascular environment and the traditional in vitro tissue culture polystyrene (TCP) substrates is that the basement membrane has an elastic modulus between 1 and 100 kPa, while TCP is several orders of magnitude stiffer in the GPa range. Long-term culture on this supra-physiological stiffness has been demonstrated to guide MSC production toward an osteoblastic lineage, reduce secretion of pro-regenerative/anti-inflammatory factors, and reduce proliferation rate over time, ultimately leading to senescence.

Interestingly, a direct comparison of MSCs on physiological-range-stiffness materials versus TCP suggests that MSCs have enhanced transcription and/or secretion of multiple paracrine factors on softer substrates ranging having a Young's modulus from 1 kPa to 40 kPa. This phenomenon has also been observed in three-dimensional cultures where trophic gene transcriptome from 3 kPa versus 18 kPa stiffness gels were distinct, indicating that one order of magnitude in modulus is sufficient to induce changes in expression of secreted proteins. This responsiveness of MSCs to substrate stiffness on has been shown to last beyond its time in contact with a particular surface. Recent studies have demonstrated that MSCs develop a biological mechanical memory of substrate stiffness that becomes irreversible after long durations of culture. Extended serial culture of MSCs on 1 kPa surfaces sustains the secretory properties of MSCs; however, proliferation is markedly reduced consistent with known focal adhesion signaling pathways that control proliferation, making this system non-ideal for culture expansion of highly secretory MSCs.

In each of the above culture systems, different matrix proteins or adhesive peptides can be used to support attachment of MSCs to the cell culture surfaces, therefore making the interpretation of the role of mechanical cues in combination with biochemical cues in controlling secretome and proliferation more difficult. MSCs experience mechanical forces through several cell-matrix and cell-cell interactions including, for example, stimulation of integrin and cadherin receptors, respectively. Expansion of MSCs on collagen-rich decellularized extracellular matrix (ECM) can enhance the secretion of individual paracrine factors. Transmembrane signaling through cell-cell interactions of cadherins can enhance MSC secretory capacity, making cadherin an attractive adhesive target for stimulating MSC secretome. Expression of N-cadherin can distinguish low and high therapeutic MSCs in a rat model of myocardial infarctions such that high expression of N-cadherin can be correlated with increased angiogenic growth factor secretion and improved functional outcome. Similarly, MSC aggregation, which is thought to maximize cell-cell interactions through cadherins without wishing to be bound by any particular scientific theory, can enhance the secretion of anti-inflammatory cytokines. In biomaterials, the presentation of N-cadherin-engaging ligands, such as the amino acid sequence HAVDI, in combination with integrin-engaging ligand RGD, can reduce MSC proliferation.

MSCs can also interact with glycosaminoglycan (GAG) moieties in the perivascular environment, such as hyaluronic acid (HA) and heparan sulfate. Heparan sulfate can sequester many positively charged autocrine/paracrine trophic factors, chemokines, and cytokines that participate in localized signaling with MSCs. Material culture surfaces presenting HA can improve MSC adhesion through ligation of the MSC surface receptor CD44, while heparin-mediated autocrine factor sequestration was shown to improve MSC adhesion and better maintain multipotency. Such an embodiment suggests that GAG incorporation into culture surfaces can modulate/enhance adhesion. Additionally, the presence of GAGs, such as heparin, can sequester growth/serum factors near the cells and therefore allow the reduction of concentration of expensive serum factors needed in the media

Therefore, embodiments of the present disclosure can provide cell culture surface materials with tailorable mechanical and biochemical properties as culture surfaces to improve the production of highly secretory MSC. Disclosed herein, for example, are culture substrate cell culture surfaces with stiffness orders of magnitude less than TCP (kPa range) incorporating biochemical moieties such as adhesive ligands and GAGs. Such embodiments can increase MSC secretory function and reduce replicative senescence compared to conventional culture on TCP.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter.

Embodiments of the present disclosure can provide a cell culture surface, comprising a polymer material comprising an acrylate functional group, and one or more functional compounds comprising one or more functional moieties. The one or more functional moieties can be selected based on functional goals, such as cell adherence, stimulation of specific cell signaling, increased secretory activity, and the like. Similarly, the one or more functional compounds can be selected based on their functional moieties or for other functional goals, such as preventing senescence, enhancing secretory activity, immobilizing exogenous or other cell-derived products, cellular adherence, stimulation of cell signaling, and the like. Additionally, the cell culture surface can have tunable mechanical properties, such as stiffness, Young's modulus, elastic modulus, compressive modulus, Poisson's ratio, and the like. Therefore, the composition and mechanical properties of the cell culture surface can be tuned to accommodate culturing a wide variety of cells, including, but not limited to, animal cells, human cells, stem cells, mesenchymal stromal cells (MSCs), and the like.

In some embodiments, the cell culture surface can have a compressive modulus of 10 kPa or greater (e.g., 20 kPa or greater, 30 kPa or greater, 40 kPa or greater, 50 kPa or greater, 60 kPa or greater, 70 kPa or greater, 80 kPa or greater, 90 kPa or greater, 100 kPa or greater, 110 kPa or greater, 120 kPa or greater, 130 kPa or greater, 140 kPa or greater, 150 kPa or greater, 160 kPa or greater, 170 kPa or greater, 180 kPa or greater, 190 kPa or greater, 200 kPa or greater, 250 kPa or greater, 300 kPa or greater, 350 kPa or greater, 400 kPa or greater, 450 kPa or greater, 500 kPa or greater, 550 kPa or greater, 600 kPa or greater, 650 kPa or greater, 700 kPa or greater, 750 kPa or greater, 800 kPa or greater, 850 kPa or greater, 900 kPa or greater, 910 kPa or greater, 920 kPa or greater, 930 kPa or greater, 940 kPa or greater, 950 kPa or greater, 960 kPa or greater, 970 kPa or greater, 980 kPa or greater, or 990 kPa or greater).

In some embodiments, the cell culture surface can have a compressive modulus of 1000 kPa or less (e.g., 20 kPa or less, 30 kPa or less, 40 kPa or less, 50 kPa or less, 60 kPa or less, 70 kPa or less, 80 kPa or less, 90 kPa or less, 100 kPa or less, 110 kPa or less, 120 kPa or less, 130 kPa or less, 140 kPa or less, 150 kPa or less, 160 kPa or less, 170 kPa or less, 180 kPa or less, 190 kPa or less, 200 kPa or less, 250 kPa or less, 300 kPa or less, 350 kPa or less, 400 kPa or less, 450 kPa or less, 500 kPa or less, 550 kPa or less, 600 kPa or less, 650 kPa or less, 700 kPa or less, 750 kPa or less, 800 kPa or less, 850 kPa or less, 900 kPa or less, 910 kPa or less, 920 kPa or less, 930 kPa or less, 940 kPa or less, 950 kPa or less, 960 kPa or less, 970 kPa or less, 980 kPa or less, or 990 kPa or less).

In some embodiments, the cell culture surface can have a compressive modulus from 10 kPa to 1000 kPa (e.g., from 20 kPa to 990 kPa, from 30 kPa to 980 kPa, from 40 kPa to 970 kPa, from 50 kPa to 960 kPa, from 60 kPa to 950 kPa, from 70 kPa to 940 kPa, from 80 kPa to 930 kPa, from 90 kPa to 920 kPa, from 100 kPa to 910 kPa, from 100 kPa to 900 kPa, from 150 kPa to 850 kPa, from 200 kPa to 800 kPa, from 250 kPa to 750 kPa, from 300 kPa to 700 kPa, from 350 kPa to 650 kPa, from 400 kPa to 600 kPa, from 450 kPa to 550 kPa, from 10 kPa to 900 kPa, from 10 kPa to 800 kPa, from 10 kPa to 700 kPa, from 10 kPa to 600 kPa, from 10 kPa to 500 kPa, from 10 kPa to 400 kPa, from 10 kPa to 300 kPa, from 10 kPa to 200 kPa, from 10 kPa to 100 kPa, from 20 kPa to 100 kPa, or from 30 kPa to 100 kPa).

In some embodiments, the polymer material can be any polymer having properties including, but not limited to, hydrophilic, flexible, and/or crosslinkable. For example, the polymer material can comprise poly(ethylene glycol) diacrylate (PEG-DA). It is understood that other hydrophilic, flexible, and/or crosslinkable polymers can be used in the polymer material. Additionally, a combination of polymers can be present in the polymer material to confer desired properties to the cell culture surface. For example, they polymer material can be selected to confer a desired stiffness (i.e., compressive modulus) to the cell culture surface.

In some embodiments, the polymer material can comprise a polymer having a number average molecular weight of 500 Da or greater (e.g., 600 Da or greater, 700 Da or greater, 800 Da or greater, 900 Da or greater, 1000 Da or greater, 1100 Da or greater, 1200 Da or greater, 1300 Da or greater, 1400 Da or greater, 1500 Da or greater, 1600 Da or greater, 1700 Da or greater, 1800 Da or greater, 1900 Da or greater, 2000 Da or greater, 2100 Da or greater, 2200 Da or greater, 2300 Da or greater, 2400 Da or greater, 2500 Da or greater, 2600 Da or greater, 2700 Da or greater, 2800 Da or greater, 2900 Da or greater, 3000 Da or greater, 3100 Da or greater, 3200 Da or greater, 3300 Da or greater, 3400 Da or greater, 3500 Da or greater, 3600 Da or greater, 3700 Da or greater, 3800 Da or greater, 3900 Da or greater, 4000 Da or greater, 4500 Da or greater, 5000 Da or greater, 5500 Da or greater, 6000 Da or greater, 6500 Da or greater, 7000 Da or greater, 7100 Da or greater, 7200 Da or greater, 7300 Da or greater, 7400 Da or greater, 7500 Da or greater, 7600 Da or greater, 7700 Da or greater, 7800 Da or greater, 7900 Da or greater, 8000 Da or greater, 8100 Da or greater, 8200 Da or greater, 8300 Da or greater, 8400 Da or greater, 8500 Da or greater, 8600 Da or greater, 8700 Da or greater, 8800 Da or greater, 8900 Da or greater, 9000 Da or greater, 9100 Da or greater, 9200 Da or greater, 9300 Da or greater, 9400 Da or greater, 9500 Da or greater, 9600 Da or greater, 9700 Da or greater, 9800 Da or greater, or 9900 Da or greater).

In some embodiments, the polymer material can comprise a polymer having a number average molecular weight of 10000 Da or less (e.g., 600 Da or less, 700 Da or less, 800 Da or less, 900 Da or less, 1000 Da or less, 1100 Da or less, 1200 Da or less, 1300 Da or less, 1400 Da or less, 1500 Da or less, 1600 Da or less, 1700 Da or less, 1800 Da or less, 1900 Da or less, 2000 Da or less, 2100 Da or less, 2200 Da or less, 2300 Da or less, 2400 Da or less, 2500 Da or less, 2600 Da or less, 2700 Da or less, 2800 Da or less, 2900 Da or less, 3000 Da or less, 3100 Da or less, 3200 Da or less, 3300 Da or less, 3400 Da or less, 3500 Da or less, 3600 Da or less, 3700 Da or less, 3800 Da or less, 3900 Da or less, 4000 Da or less, 4500 Da or less, 5000 Da or less, 5500 Da or less, 6000 Da or less, 6500 Da or less, 7000 Da or less, 7100 Da or less, 7200 Da or less, 7300 Da or less, 7400 Da or less, 7500 Da or less, 7600 Da or less, 7700 Da or less, 7800 Da or less, 7900 Da or less, 8000 Da or less, 8100 Da or less, 8200 Da or less, 8300 Da or less, 8400 Da or less, 8500 Da or less, 8600 Da or less, 8700 Da or less, 8800 Da or less, 8900 Da or less, 9000 Da or less, 9100 Da or less, 9200 Da or less, 9300 Da or less, 9400 Da or less, 9500 Da or less, 9600 Da or less, 9700 Da or less, 9800 Da or less, or 9900 Da or less).

In some embodiments, the polymer material can comprise a polymer having a number average molecular weight from 500 Da to 10000 Da (e.g., from 600 Da to 9900 Da, from 700 Da to 9800 Da, from 800 Da to 9700 Da, from 900 Da to 9600 Da, from 1000 Da to 9500 Da, from 1100 Da to 9400 Da, from 1200 Da to 9300 Da, from 1300 Da to 9200 Da, from 1400 Da to 9100 Da, from 1500 Da to 9000 Da, from 1600 Da to 8900 Da, from 1700 Da to 8800 Da, from 1800 Da to 8700 Da, from 1900 Da to 8600 Da, from 2000 Da to 8500 Da, from 2100 Da to 8400 Da, from 2200 Da to 8300 Da, from 2300 Da to 8200 Da, from 2400 Da to 8100 Da, from 2500 Da to 8000 Da, from 2600 Da to 7900 Da, from 2700 Da to 7800 Da, from 2800 Da to 7700 Da, from 2900 Da to 7600 Da, from 3000 Da to 7500 Da, from 3100 Da to 7400 Da, from 3200 Da to 7300 Da, from 3300 Da to 7200 Da, from 3400 Da to 7100 Da, from 3500 Da to 7000 Da, from 600 Da to 3400 Da, from 700 Da to 3300 Da, from 800 Da to 3200 Da, from 900 Da to 3100 Da, from 1000 Da to 3000 Da, from 1100 Da to 2900 Da, from 1200 Da to 2800 Da, from 1300 Da to 2700 Da, from 1400 Da to 2600 Da, or from 1500 Da to 2500 Da).

In some embodiments, the polymer material can be present in the cell culture surface in a concentration of 1% or greater (e.g., 2% or greater, 3% or greater, 4% or greater, 5% or greater, 6% or greater, 7% or greater, 8% or greater, 9% or greater, 10% or greater, 11% or greater, 12% or greater, 13% or greater, 14% or greater, 15% or greater, 16% or greater, 17% or greater, 18% or greater, 19% or greater, 20% or greater, 21% or greater, 22% or greater, 23% or greater, 24% or greater, 25% or greater, 30% or greater, 35% or greater, 40% or greater, 45% or greater, 50% or greater, 55% or greater, 60% or greater, 65% or greater, or 70% or greater) by weight per unit volume of cell culture surface.

In some embodiments, the polymer material can be present in the cell culture surface in a concentration of 75% or less (e.g., 2% or less, 3% or less, 4% or less, 5% or less, 6% or less, 7% or less, 8% or less, 9% or less, 10% or less, 11% or less, 12% or less, 13% or less, 14% or less, 15% or less, 16% or less, 17% or less, 18% or less, 19% or less, 20% or less, 21% or less, 22% or less, 23% or less, 24% or less, 25% or less, 30% or less, 35% or less, 40% or less, 45% or less, 50% or less, 55% or less, 60% or less, 65% or less, or 70% or less) by weight per unit volume of cell culture surface.

In some embodiments, the polymer material can be present in the cell culture surface in a concentration from 1% to 75% (e.g., from 2% to 70%, from 3% to 65%, from 4% to 60%, from 5% to 55%, from 6% to 50%, from 1% to 50%, from 1% to 45%, from 1% to 40%, from 1% to 35%, from 1% to 30%, from 1% to 25%, from 2% to 25%, from 3% to 25%, from 4% to 25%, from 5% to 25%, from 6% to 24%, from 7% to 23%, from 8% to 22%, from 8% to 21%, from 8% to 20%, from 9% to 19%, from 10% to 18%, from 11% to 17%, or from 12% to 16%) by weight per unit volume of cell culture surface.

In some embodiments, the one or more functional compounds can comprise one or more functional moieties selected to tune the cell culture surface and/or confer desired properties to the cell culture surface. For instance, the cell culture surfaces can be functionalized with various moieties to modulate cell adhesion, proliferation, and secretory activity. The one or more functional compounds can comprise functional groups such as peptides or proteins derived from extra-cellular matrix components along with other moieties used to encourage cell adhesion. Such functional groups can also include peptide/proteins or antibodies that are meant to interact with specific cell-surface receptors, such as cadherin and integrin-engaging motifs. The one or more functional compounds can also include carbohydrates and glycosaminoglycans (GAGs), such as heparins, chondroitin sulfate, hyaluronic acids, desulfated heparins, linear repeating disaccharides, and any derivatives of the aforementioned functional compounds that are capable of modulating the secretion and sequestration of multiple factors by mesenchymal stem cells. Additionally, such functional groups may also include lipid moieties that are capable of interacting with various cell receptors in a manner that can either modulate cell adhesion, proliferation, or secretion, as well as promote the sequestration of secreted factors. As one or ordinary skill in the art would appreciate, the amount of the one or more functional compounds may vary depending on the desired properties wished to be conferred to the cell culture surface.

Therefore, the embodiments of the present disclosure can provide a set of cell culture surface formulations to control the secretory behavior of cells in contact with the cell culture surface. An advantage of the present disclosure over existing cell culture technology is the ability to control the secretion of various immunomodulatory and regenerative factors of cells in contact with the gel by altering the mechanical and “biologically active” properties of the gel matrix. Some cell culture surfaces as disclosed herein can be used to amplify the pro-regenerative secretome of multiple somatic cells, including any adhesion-dependent cell, as well as stem/progenitor cells, including but not limited to mesenchymal stem/stromal cells, hematopoietic stem cells, embryonic stem cells, and pluripotent stem cells. Cells can be culture expanded and released from these cell culture surfaces in a similar fashion to traditional cell culture substrates.

Embodiments of the present disclosure can also, therefore, include a set of cell culture surface formulations to modulate the sequestration of cell-secreted proteins within the gel. An advantage of the present disclosure over existing cell culture technologies is the ability to control the degree of sequestration of proteins secreted by cells in contact with the gel by altering the incorporation of functional moieties within the gel matrix. Some cell culture surfaces as disclosed herein can modulate protein sequestration and can be used to retain cell-secreted proteins within the gel and therefore in proximity to the cell surface for paracrine signaling purposes. Local paracrine signaling via protein sequestration of cell culture surfaces may be used to encourage adhesion and proliferation of cells in contact with cell culture surfaces, as well as for regulation of cell phenotype. Such cell culture surfaces can be used to enhance proliferation and/or function of multiple somatic cells, including any adhesion-dependent cell, as well as stem/progenitor cells, including but not limited to mesenchymal stem cells, hematopoietic stem cells, and pluripotent stem cells. Cells can be culture expanded and released from these cell culture surfaces in a similar fashion to traditional cell-culture substrates.

Embodiments of the present disclosure can also, therefore, include a set of cell culture surface formulations to support the growth of cells in the absence of serum or significantly-reduced serum culture conditions. An advantage of the present disclosure over existing cell culture technologies is the ability to improve levels of cell proliferation with significantly reduced-levels of serum content. Such an embodiment can overcome a major cell manufacturing hurdle by eliminating the dependence on serum and recombinant growth factors for cell culture and expansion. Cell culture surfaces with specific mechanical properties and motif incorporation can retain cell-secreted proteins within the gel for paracrine signaling that promotes cell proliferation in the absence of serum or recombinant growth factors. These cell culture surfaces can be used to promote serum-free growth of multiple somatic cells, including any adhesion-dependent cell, as well as stem/progenitor cells, including but not limited to mesenchymal stem cells, hematopoietic stem cells, and pluripotent stem cells. Cells can be culture expanded and released from these cell culture surfaces in low-serum or serum-free cultures.

Additionally, the cell culture surface formulations as disclosed herein can also be used to reduce cellular senescence compared to traditional cell culture substrates.

It is also to be understood that the cell culture surfaces as disclosed herein can be used to culture cells followed by in vivo injection for further culturing.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

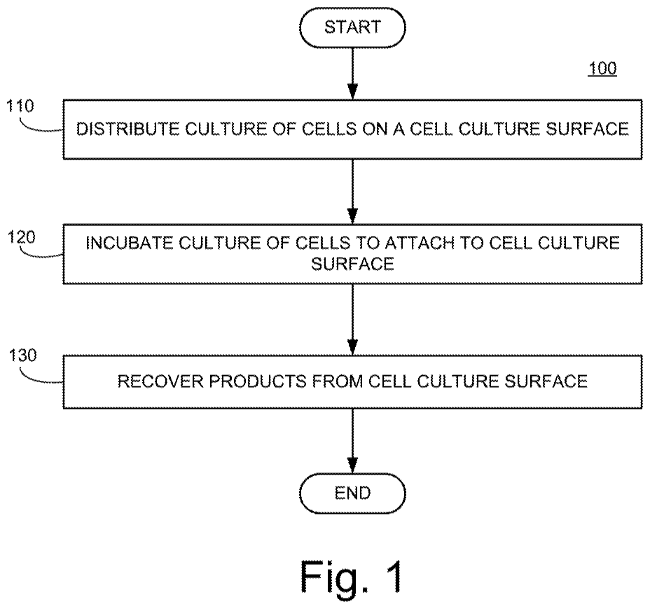

FIG. 1 illustrates a flowchart of an exemplary method 100 for culturing cells using a cell culture surface according to some embodiments of the present disclosure. As shown, in block 110, a culture of animal cells can be distributed on a cell culture surface. In some embodiments, the cell culture surface can comprise a polymer material comprising an acrylate functional group and one or more functional compounds having one or more functional moieties. Additionally, the cell culture surface can have a compressive modulus from 10 kPa to 1000 kPa.

The culture of animal cells can be distributed on a surface of the cell culture surface. In some embodiments, the cell culture surfaces can be formed as flat surfaces in bioreactor systems for cell expansion or culture to prime cells before they are used for a specific application. Cell culture surfaces can also be formed to exhibit nanopatterned surfaces in order to further alter cell secretory activity. Additionally, cell culture surfaces can be formed into microparticles or other three-dimensional shapes as cell carriers for their use in bioreactors systems or as delivery vehicles for cell therapies. Cell culture surfaces may also be formed as coatings or linings for medical devices and bioreactor systems. In some embodiments, method 100 can then proceed to block 120.

In block 120, the culture of animal cells can be incubated such that the culture of animal cells attaches to the cell culture surface. The culture of animal cells can have an initial quantity of cells being cultured that can increase during culturing. For example, the culture of animal cells can be cultured from 1 day to 10 days (e.g., from 2 days to 10 days, from 3 days to 10 days, from 4 days to 10 days, from 5 days to 10 days, from 1 day to 5 days, from 1 day to 4 days, from 1 day to 3 days, or from 1 day to 2 days). Other lengths of time for culturing are considered and may be changed as desired by one of skill in the art.

In some embodiments, after culturing, the initial quantity of cells being cultured can increase by a factor of 1 or greater (e.g., 1.5 or greater, 2 or greater, 2.5 or greater, 3 or greater, 3.5 or greater, 4 or greater, 4.5 or greater, 5 or greater, 5.5 or greater, 6 or greater, 6.5 or greater, 7 or greater, 7.5 or greater, 8 or greater, 8.5 or greater, 9 or greater or 9.5 or greater) over the course of 4 days of culturing. In some embodiments, after culturing, the initial quantity of cells being cultured can increase by a factor of 10 or less (e.g., 1.5 or less, 2 or less, 2.5 or less, 3 or less, 3.5 or less, 4 or less, 4.5 or less, 5 or less, 5.5 or less, 6 or less, 6.5 or less, 7 or less, 7.5 or less, 8 or less, 8.5 or less, 9 or less or 9.5 or less) over the course of 4 days of culturing. In some embodiments, after culturing, the initial quantity of cells being cultured can increase by a factor from 1 to 10 (e.g., from 1.5 to 9.5, from 2 to 8, from 2.5 to 7.5, from 3 to 7, from 3.5 to 6.5, or from 4 to 6) over the course of 4 days of culturing. In some embodiments, method 100 can then proceed to block 130.

In block 130, the culture of animal cells can be recovered from the cell culture surface. In some embodiments, the culture media can be recovered to obtain cell-derived products. The recovered products from the cell culture surface and/or the culture media can be purified using techniques such as column chromatography, centrifugation, filtration, and the like. In some embodiments, method 100 may terminate and complete after block 130. However, in other embodiments, the method may continue on to other method steps not shown.

For example, during the incubating (i.e., block 120) the initial quantity of cells being cultured can be reseeded to a new cell culture surface. The new cell culture surface can have substantially similar properties to the first cell culture surface; however, the new cell culture surface can have different properties to increase desired aspects of the cells being cultured. For instance, a first cell culture surface can be formulated with a composition to improve proliferation, and the cultured cells can be reseeded to a second cell culture surface formulated with a composition to increase the secreted factors. In such an embodiment, a “cascade” of cell culture surfaces can be provided by reseeding the cultured cells to enhance certain properties of the cell cultures.

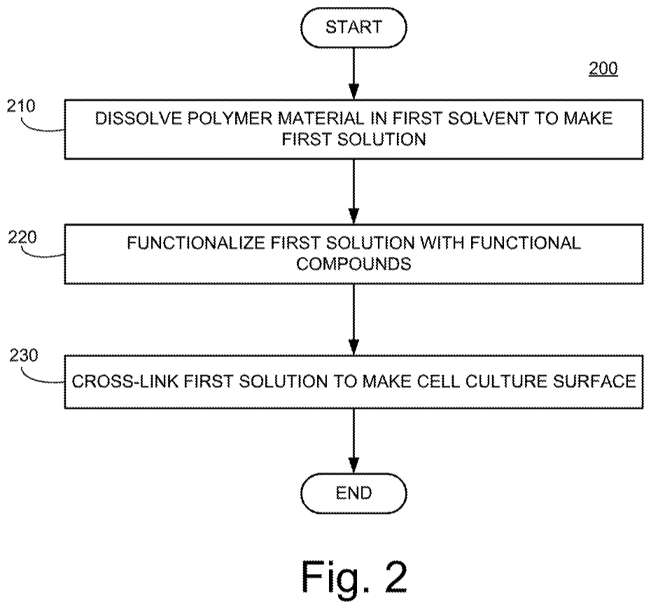

FIG. 2 illustrates a flowchart of an exemplary method 200 for making a cell culture surface according to some embodiments of the present disclosure. For example, embodiments of the present disclosure can provide cell culture surfaces comprising a poly(ethylene glycol diacrylate) (PEG-DA) backbone and interchangeable methacrylate or acrylate functional groups. The gels can be polymerized using mechanisms such as free radical initiation or photoinitiation. Hydrolytic degradation can be controlled by the concentration of 1,4-dithiothreitol (DTT) incorporated into the matrix. Proteolytic degradability can be controlled by the addition of a peptide linker with protease cleavable site. The cell culture surfaces can be tailored to exhibit a range of mechanical properties based on the factors that compose the cell culture surface, as described above, including the length and amount of polymer material included in the network, the concentration of free radical initiators, or the curing time. This method of creating cell culture surfaces suitable for cell culture with tailorable mechanical properties be used for the formation of surfaces with other acrylated polymeric biomolecules.

As shown, in block 210, the polymer material can be dissolved in a first solvent to create a first solution. As discussed, the polymer material can comprise an acrylate functional group. Embodiments of a polymer material are discussed above. The first solvent can be any solvent capable of dissolving the polymer material to create a substantially homogeneous solution. Additionally, the first solvent may be selected to confer desired properties to the first solution. In some embodiments, method 200 can then proceed to block 220.

In block 220, the first solution can be functionalized with one or more functional compounds comprising one or more functional moieties. Embodiments of the one or more functional compounds and the one or more functional moieties are described above. In some embodiments, at least one moiety from the one or more functional moieties can be configured to adhere to animal cells for culturing. In some embodiments, the functionalizing can comprise adding the one or more functional compounds to the first solution via mixing, stirring, dissolving, and the like. In some embodiments, method 200 can then proceed to block 230.

In block 230, the first solution can be cross-linked to obtain a cell culture surface. In some embodiments, the cross-linked cell culture surface can have a compressive modulus from 10 kPa to 1000 kPa. The cross-linking can be accomplished through a variety of methods. In some embodiments, method 200 may terminate and complete after block 230. However, in other embodiments, the method may continue on to other method steps not shown.

Certain embodiments and implementations of the disclosed technology are described above with reference to block and flow diagrams of systems and methods according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

### Examples

The following examples are provided by way of illustration but not by way of limitation.

Poly(ethylene glycol) diacrylate (PEG-DA, 3.4 kDa average) and fully desulfated heparin (Hep⁻) can be prepared. Briefly, heparin sodium salt from porcine intestinal mucosa (Hep, Sigma-Aldrich) can be dissolved in H₂O and passed through a Dowex 50WX4 resin (mesh size 100-200, Sigma-Aldrich). Pyridine can be added until the solution reaches approximately pH 6, after which excess pyridine can be removed via rotary evaporator. The remaining solution can be flash frozen and lyophilized to form dry heparin pyridinium, which can then be dissolved at 10 mg/mL in a 9:1 v/v N-methylpyrrolidone (NMP)/H₂O solution at 100° C. for 24 hours. The resulting Hep⁻ can then be precipitated with 95% ethanol saturated sodium acetate, collected via centrifugation, and dissolved in H₂O. Lastly, the Hep⁻ solution can be dialyzed, lyophilized, and stored at −20° C.

Hep, Hep⁻ and HA (Lifecore Biomedical) can be separately functionalized with methacrylamide. Each GAG can be reacted with approximately 2.5 to 3-fold molar excess of N-hydroxysulfosuccinimide sodium salt (Sigma), N-(3-aminopropyl) methacrylamide hydrochloride (Polysciences) and (N-3-Dimethylaminopropyl)-N′-ethylcarbodiimide hyrdochloride (EDC) (Sigma-Aldrich) in a phosphate buffer of pH 5.5 on ice. After 2 hours, additional EDC can be added with continued stirring for 4 hours. The solution can be dialyzed against deionized water for 3 days using 3.5 kDa molecular weight cutoff dialysis tubing (Spectrum) and lyophilized. All polymers can be purged under nitrogen gas and stored at −20° C., protected from light prior to use.

Cell-adhesive peptides can be conjugated to linear acrylate-PEG spacers. An integrin-engaging peptide GRGDS (Bachem) can be dissolved in 50 mM sodium bicarbonate buffer at pH 8.5. Acryl-PEG-succinimidyl valerate (Acryl-PEG-SVA, Mₙ˜3.4 kDa, Laysan Bio) can be added at a molar ratio of 1:2 peptide:Acrl-PEG-SVA. After reacting for 3 hours with gentile stirring, the solution can be dialyzed against H₂O using 3.5 kDa molecular weight cutoff tubing for 2 days and lyophilized. Similarly, an N-cadherin-engaging peptide HAVDI (HAVDIGGGC, Genscript) can be dissolved in phosphate buffer saline (PBS, Teknova) and Acryl-PEG-maleimide (Acryl-PEG-Mal, Mₙ˜3.4 kDa, Laysan Bio) can be added at a molar ratio of 1:2 peptide:Acrl-PEG-Mal. After reacting for 3 hours with gentile stirring, the solution can be dialyzed against H₂O for 2 days and lyophilized. Acryl-PEG-GRGDS and Acryl-PEG-HAVDI can be purged under nitrogen gas and stored at −20° C., protected from light prior to use.

Cell culture surfaces can be prepared with a range of mechanical properties in order to characterize the mechanical and biochemical properties of the materials. PEG-DA (3.4 kDa or 600 Da) can be dissolved in PBS to create polymer solution of a final concentration from 8 to 20% wt/v. Cross-linking can be accomplished by addition of ammonium persulfate (APS, Sigma-Aldrich) and tetramethyl ethylene diamine (TEMED, Bio Rad) at a final molarity of 18 mM. The cell culture surface precursor solution can then be immediately injected in between glass slides (spacer thickness=1 mm). After 20 minutes of cross-linking, cell culture surfaces disks can be cut from the slab using 8 mm diameter biopsy punches and the gels can be incubated in PBS overnight at 37° C. to reach equilibrium swelling. Undefined compression testing was performed. A Bose 3200 instrument can be programmed to compress cell culture surface substrates at a constant rate of displacement (0.1 mm/sec). The measured displacement and corresponding compressive force can be recorded at a sampling rate of 10 Hz. Stress can be calculated by dividing the compressive force by the surface area of the gel, which can be measured using calipers. The region of the resulting stress-strain curve from 10 to 15% strain can be fit to a line, and the slope can be approximated as the compressive modulus. The swelling ratio of the cell culture surfaces can also be characterized by measurements of wet weight and/or dry weight. After overnight equilibrium swelling, the weights of swollen cell culture surfaces can be recorded, and the gels can be subsequently lyophilized for 3-4 hours to obtain the dry weight. Respective compressive moduli and swelling ratio of cell culture surfaces composed of specific formulations can then be compared to one another. Fluorescamine- and dimethylmethylene blue (DMMB)-based assays can be performed to demonstrate that cell culture surfaces with different stiffnesses and swelling properties did not alter adhesive ligand and GAG density, respectively. For the fluorescamine-based assay, 30 kPa and 100 kPa cell culture surfaces can be formed with densities of Acryl-PEG-NH₂ (Creative PEGWorks) ranging from 0 to 5 mM and can be incubated overnight in PBS at 37° C. Cell culture surfaces can then be formed into 6 mm diameter disks using a biopsy punch and then placed in a black 96-well plate. Cell culture surfaces can then be incubated in 3 mg/mL fluorescamine dissolved in acetone for 10 minutes, then analyzed on a plate reader for fluorescence (365 nm excitation, 460 nm emission filters). For the DMMB assay, 30 kPa and 100 kPa cell culture surfaces can be formed with Hep content ranging from 0 to 4 wt % and can be incubated overnight in PBS at 37° C. Cell culture surfaces can then be formed into 6 mm diameter disks using a biopsy punch and then placed in a clear 96-well plate. Cell culture surface disks can then be incubated for 60 minutes in 16 mg/L DMMB in H₂O with 40 mM glycine (Sigma-Aldrich), 40 mM NaCl (Sigma-Aldrich) and 9.5 mM HCl. Afterwards, the gels can be removed from DMMB solution, then analyzed on a plate reader for absorbance at 520 nm.

Human bone marrow MSCs (RoosterBio, Inc.) can be culture expanded for two population doublings after received and frozen in ˜5×10⁵ cell aliquots in CryoStorCS10 freezing media (BioLife). For all experiments, frozen aliquots can be plated on tissue culture polystyrene for 3-4 days prior to seeding onto test surfaces. MSCs can be maintained in low glucose Dulbecco's Minimal Essential Medium (DMEM, 1 g/L glucose, sodium pyruvate, L-glutamine, Gibco) supplemented with 10% fetal bovine serum (FBS, Atlanta Biologicals, lot E16063) and 1% antibiotic-antimycotic (Gibco). TrypLE express (Gibco) can be used to enzymatically passage MSCs and to lift cells from cell culture surfaces. Donor information supplied by manufacturer: Donor 1: Lot 139, Female, 26 years old, 9.4 population doublings upon receipt; Donor 2: Lot 182, Male, 25 years old, 8.9 population doublings upon receipt.

Polymer components can be sterile filtered using Spin-X centrifuge tube filters (Costar) or other methods, such as syringe filter units, prior to cross-linking. For secretome characterization, 12 mm diameter, 0.5 mm thickness cell culture surface discs can be prepared and placed in ultra-low attachment 24 well plates (Corning) overnight with PBS to allow swelling and removal of leachable products. Fabricated cell culture surfaces can then be washed once with PBS and once with complete media prior to seeding cells. MSCs can be diluted to 375,000 cells/mL in complete media and 40 uL can be placed directly on top of each cell culture surface or TCP well to seed 15,000 cells per cell culture surface disc or well.

Cells can be incubated at 37° C. for 10-20 minutes to allow initial attachment prior to bringing the final media volume to 500 uL media. For serial culture on cell culture surfaces, cells can be maintained on surfaces for four days, then passaged to freshly prepared surfaces as described. For senescence analysis, cells can be maintained for 3 successive passages on 100 kPa cell culture surfaces (12 days). For secretome studies, cells can be collected from TCP or surfaces by TrypLE Express dissociation and frozen for Quant-it Picogreen DNA analysis (Invitrogen).

MSCs can be plated on cell culture surfaces or TCP and incubated for 4 days in a standard cell culture incubator to generate conditioned media (CM). At endpoint, media can be collected for analysis and centrifuged at 1,000×g to remove any potential cell debris before freezing the supernatant at −80 C in aliquots until use. CM can be evaluated by Luminex Multiplex ELISA assay for 41 human chemokines and cytokines (HCYTOMAG-60K, EMD Millipore) and analyzed by integrated Milliplex Analyst software. Background levels of these factors from complete media can be subtracted from final values and factors can be normalized by the number of cells in the sample.

Senescent cells can be identified by senescence associate β-galactosidase staining following manufacturer protocol (Cell Signaling Technology #9860). Briefly, after 4, 8, or 12 days of culture on either TCP or RGD 100 kPa cell culture surfaces, cells can be washed, fixed, and then incubated with staining solution overnight at 37° C. Staining solution can be removed, cells can be washed with PBS, and nuclei can be stained with Hoechst dye to identify total cell number. Cells can be imaged at 20× magnification and the number of β-gal+ cells can be recorded as a percent of total cells.

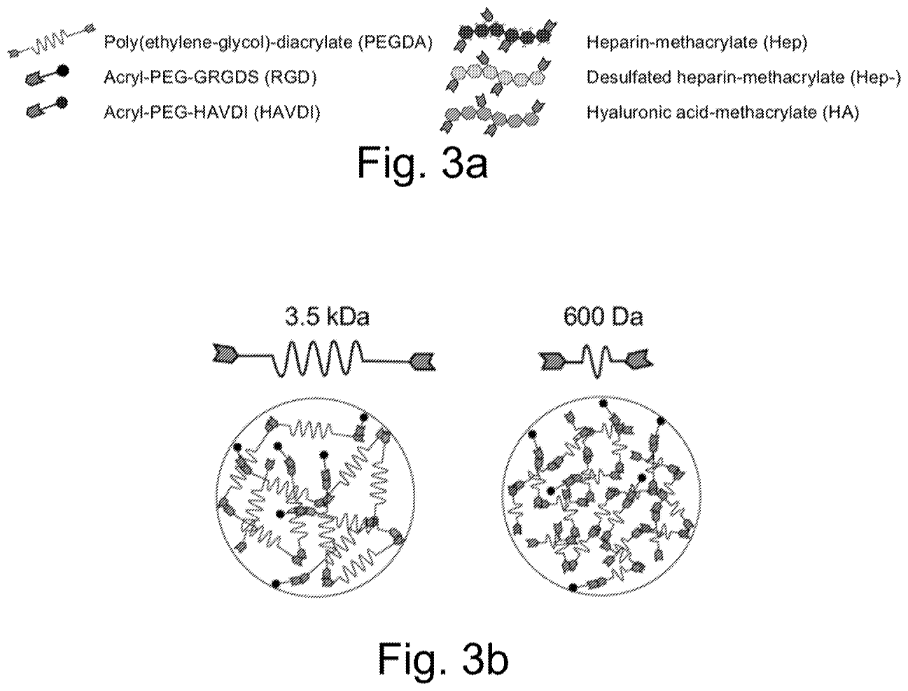

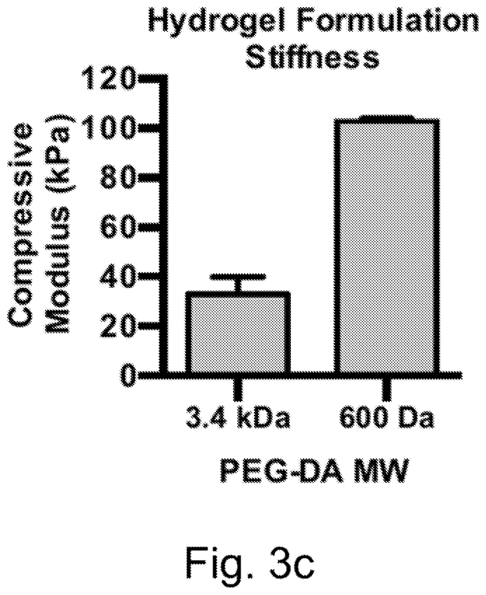

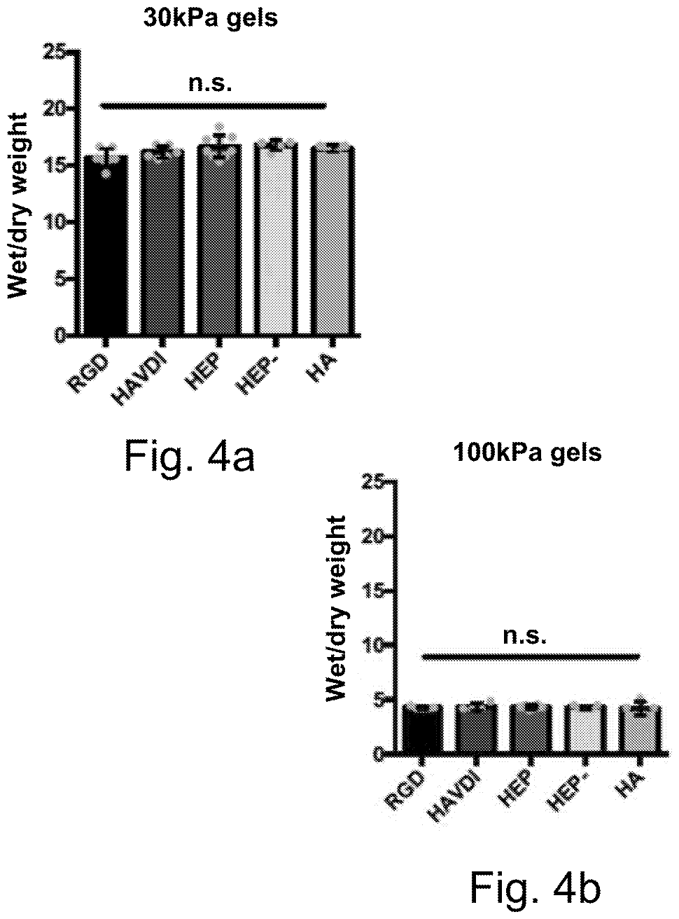

In order to screen the combined effect of mechanical properties and biomolecule presentation on MSC-secreted factors and MSC growth, PEG-DA-based culture substrates can be synthesized with different combinations of adhesive ligands, GAGS, and mechanical properties, examples of which can be found in FIG. 3a. Cell culture surface stiffness can be modulated by varying polymer molecular weight (3.4 kDa or 600 Da) and final concentration of PEG-DA, as shown in FIG. 3b. Cell culture surfaces made from 8.5 wt % 3.4 kDa PEG-DA can exhibit a compressive modulus of 33.0±6.8 kPa and 20 wt % 600 Da PEG-DA formulations can exhibit a modulus of 102.4±1.1 kPa, hereafter referred to as 30 kPa and 100 kPa, respectively as shown in FIG. 3c. The ratio of swollen polymer to dry polymer weight (swelling ratio) can be used to identify specific initiator concentrations necessary for each formulation to normalize substrate modulus across 30 kPa and 100 kPa gels functionalized with adhesive ligands and GAGs, as shown in FIG. 4a and FIG. 4b. Specific examples of functionalized formulations are illustrated below in Table I.

TABLE I
Exemplary embodiments of cell culture surface formulations

| | RGD | | HAVDI | | Hep | | Hep− | | HA | |
|---|---|---|---|---|---|---|---|---|---|---|
| 30 kPa | | | | | | | | | | |
| Total polymer wt % | 8.5% | (wt/v) | 8.5% | (wt/v) | 8.5% | (wt/v) | 8.5% | (wt/v) | 8.5% | (wt/v) |
| PEGDA 3.4 kDa | 8.17% | (wt/v) | 7.72% | (wt/v) | 6.61% | (wt/v) | 6.61% | (wt/v) | 6.61% | (wt/v) |
| Acryl-PEG-GRGDS | 3.89 | mg/mL | 3.89 | mg/mL | 3.89 | mg/mL | 3.89 | mg/mL | 3.89 | mg/mL |
| Acryl-PEG-HAVDI | | | 3.89 | mg/mL | | | | | | |
| MA-Hep | | | | | 15 | mg/mL | | | | |
| MA-Hep− | | | | | | | 15 | mg/mL | | |
| MA-HA | | | | | | | | | 15 | mg/mL |
| TEMED | 0.018 | mM | 0.018 | mM | 0.024 | mM | 0.018 | mM | 0.018 | mM |
| APS | 0.018 | mM | 0.018 | mM | 0.024 | mM | 0.018 | mM | 0.018 | mM |
| 100 kPa | | | | | | | | | | |
| Total polymer wt % | 20% | (wt/v) | 20% | (wt/v) | 20% | (wt/v) | 20% | (wt/v) | 20% | (wt/v) |
| PEGDA 600 Da | 19.61% | (wt/v) | 19.22% | (wt/v) | 18.11% | (wt/v) | 18.11% | (wt/v) | 18.11% | (wt/v) |
| Acryl-PEG-GRGDS | 3.89 | mg/mL | 3.89 | mg/mL | 3.89 | mg/mL | 3.89 | mg/mL | 3.89 | mg/mL |
| Acryl-PEG-HAVDI | | | 3.89 | mg/mL | | | | | | |
| MA-Hep | | | | | 15 | mg/mL | | | | |
| MA-Hep− | | | | | | | 15 | mg/mL | | |
| MA-HA | | | | | | | | | 15 | mg/mL |
| TEMED | 0.018 | mM | 0.018 | mM | 0.024 | mM | 0.018 | mM | 0.018 | mM |
| APS | 0.018 | mM | 0.018 | mM | 0.024 | mM | 0.018 | mM | 0.018 | mM |

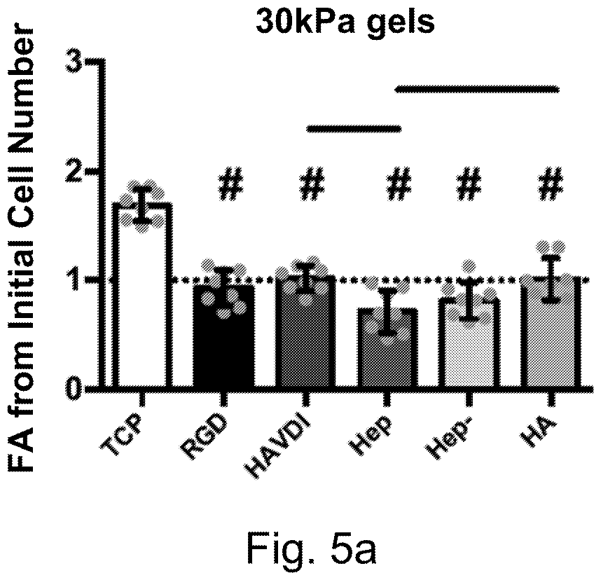

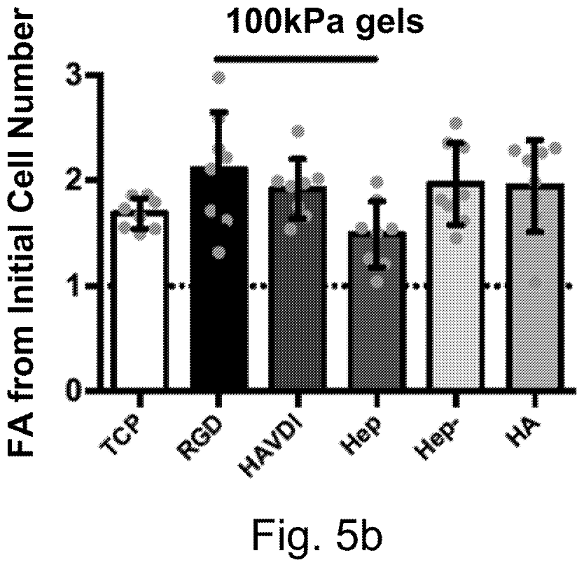

MSCs can adhere to all cell culture surface culture surfaces. After 4 days, MSCs cultured on 30 kPa surfaces, regardless of biomolecule presentation, can have a lower cell density compared to those on 100 kPa surfaces or TCP. MSCs cultured on all 100 kPa surfaces can exhibit similar morphology to that of MSCs cultured on TCP. MSC cultures on all 30 kPa surfaces can result in significantly lower final cell numbers than TCP and were not significantly different than the initial number of seeded cells, as shown in FIG. 5a. All 100 kPa cell culture surfaces can have final cell numbers similar to that of TCP, however the presentation of heparin reduced the final cell number compared to RGD only cell culture surfaces, as shown in FIG. 5b.

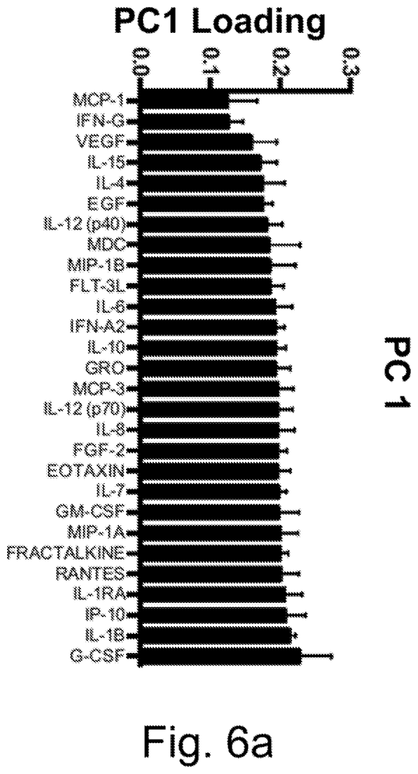

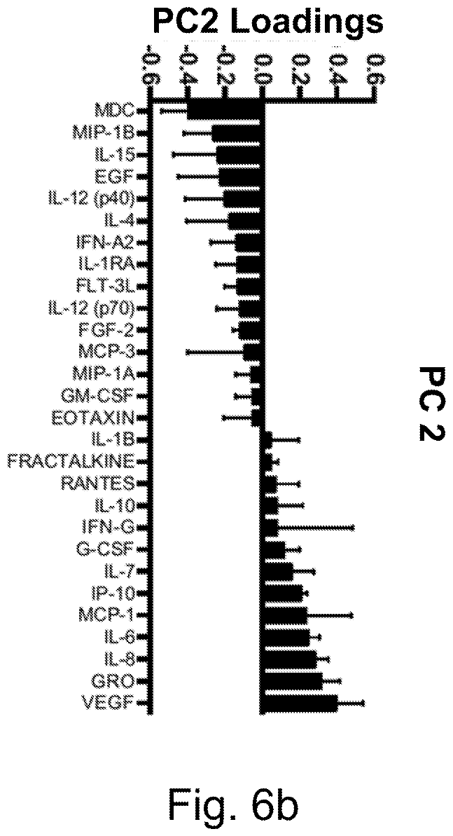

Secretome characterization can be performed on the CM of MSCs cultured on all surfaces by multiplex ELISA and normalized by cell number. MSCs cultured on all 30 kPa gels can exhibit an overall increase in abundance of immunomodulatory factor secretion versus TCP and all 100 kPa cell culture surfaces. The CM of MSCs cultured on 100 kPa Hep cell culture surfaces can exhibit a decrease of immunomodulatory factor secretion versus all surfaces which is consistent with the pulldown of these soluble factors by the Hep in the gel matrix. PCA can reveal distinct separation between secretomes of MSCs cultured on 30 kPa and 100 kPa surfaces along the first principal component (PC1), which accounted for 77% of the variance in the dataset shown in FIG. 6a and FIG. 6b. All loadings on PC1 are positive and similar in magnitude which is consistent with the observation that most of the detectable secreted factors are elevated on the 30 kPa cell culture surfaces. PCA can reveal separation of CM from MSCs cultured on 100 kPa Hep cell culture surfaces along both PC1 and PC2 as shown in FIG. 6a and FIG. 6, respectively.

Quantification of individual immunomodulatory cytokines and chemokines can demonstrate increased secretion of several factors, including VEGF, IL-6, IL-8, GRO and Fractalkine, by MSCs cultured on 30 kPa cell culture surfaces versus those cultured on TCP. Additionally, MSCs cultured on 100 kPa Hep cell culture surfaces can exhibit significant decreases in the secretion of all factors versus those cultured on TCP. Interestingly, the secretion of the inflammatory factors of monocyte chemoattractant protein 1 (MCP-1) and IL-6 can be downregulated by MSCs cultured on multiple formulations of 100 kPa cell culture surfaces versus TCP.

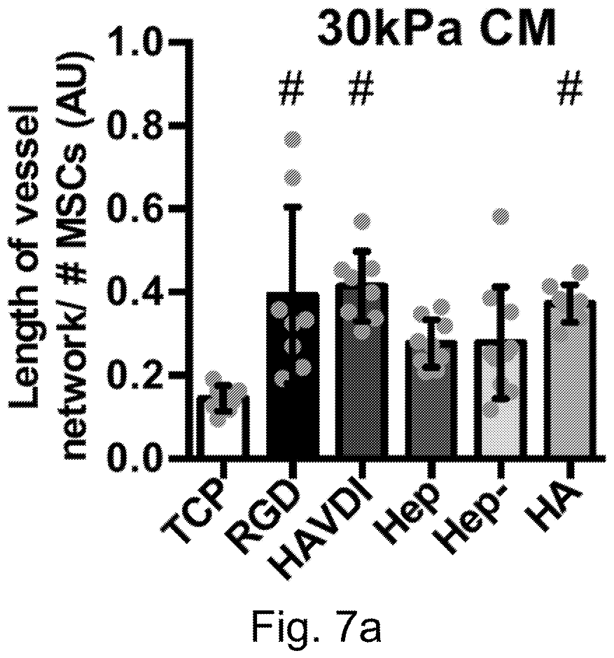

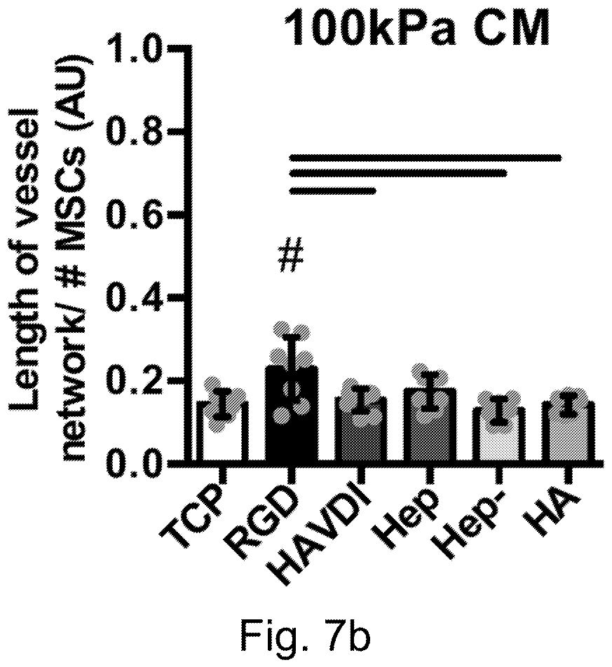

To determine whether these changes in paracrine activity result in functional changes in downstream cell types, CM from MSCs cultured on various surfaces can be used to assess changes in functional activity of endothelial cells, myeloid cells, and myoblasts. To examine the effect of MSC CM on angiogenesis, an in vitro endothelial tube formation assay can be used. HUVECs can be incubated with MSC CM, plain MSC media, or endothelial growth media for 24 hrs. CM from MSCs cultured on 30 kPa RGD, HAVDI, and HA presenting cell culture surfaces can increase HUVEC network formation compared to TCP-derived CM, as shown in FIG. 7a. CM from MSCs cultured on 100 kPa RGD gels can improve network formation compared to TCP and all other 100 kPa surfaces except Hep, as shown in FIG. 7b. CM from cultures on all 30 kPa surfaces except Hep can enhance HUVEC network formation compared to 100 kPa surfaces of the same composition (Two-way ANOVA, p<0.05, data not shown).

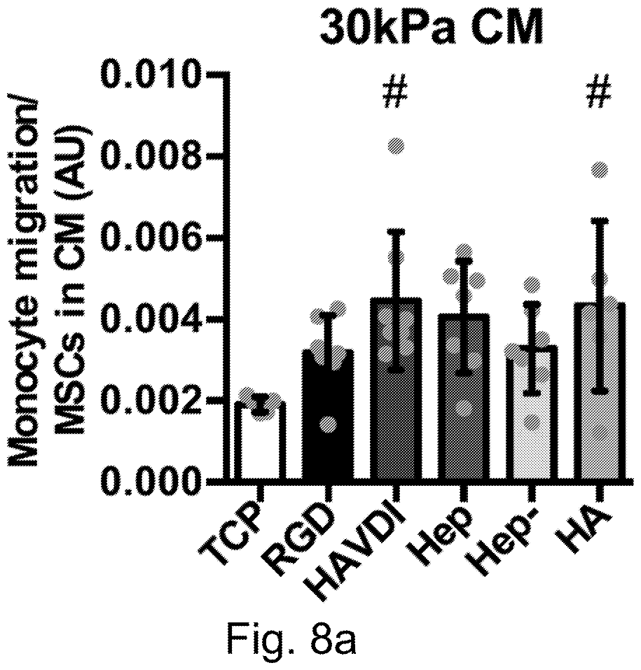

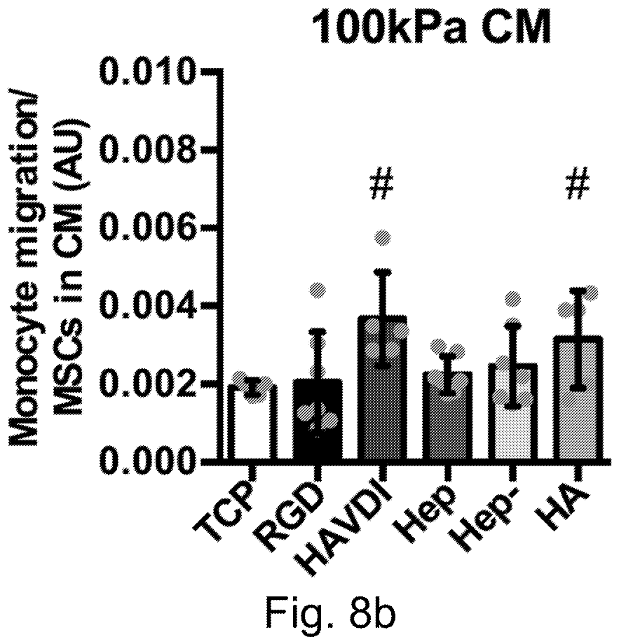

To test whether culture on cell culture surfaces can impact the chemotactic potential of MSC CM, the recruitment of serum starved THP-1 human monocytes to the CM can be tested. MSCs cultured on HAVDI- and HA-presenting surfaces of both stiffnesses can produce CM that enhanced monocyte chemotaxis compared to CM of MSCs cultured on standard TCP. An exemplary embodiment using a 30 kPa CM is shown in FIG. 8a, while an exemplary embodiment using a 100 kPa CM is shown in FIG. 8b.

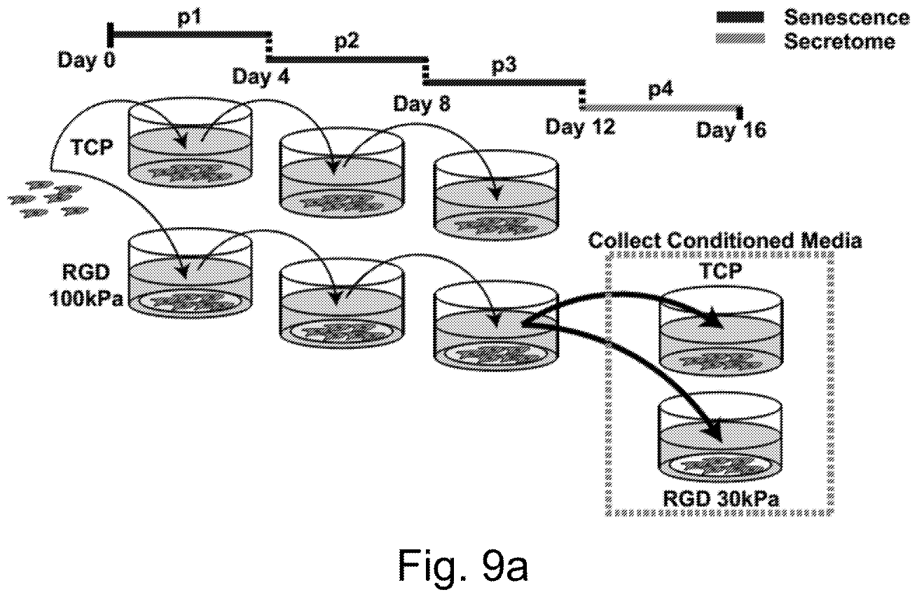

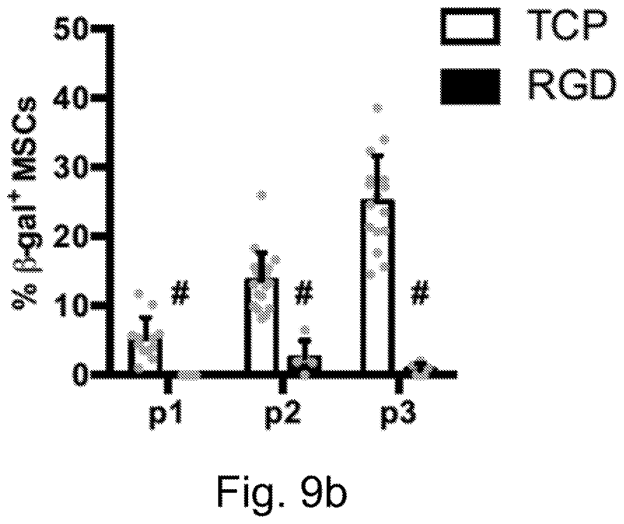

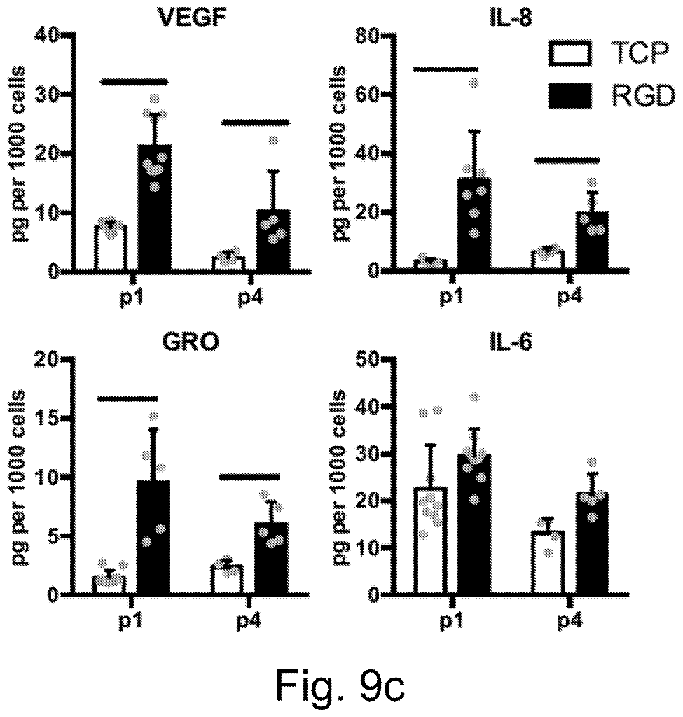

To examine the effect of culture surface properties on cell fitness in culture, serial passaging of MSCs on 100 kPa substrates can be performed and evaluated to identify changes in replicative senescence, as shown in FIG. 9a. MSCs cultured on TCP can exhibit increasing levels of senescence over three passages (p1: 5.27±3.04%, p2: 13.67±4.02%, p3: 25.06±6.60%), as shown in FIG. 9b. MSCs cultured on 100 kPa RGD cell culture surfaces can exhibit significantly lower levels of β-gal⁺ cells over the course of the successive passages, also as shown in FIG. 9b.

To determine whether the MSCs serially-passaged on 100 kPa cell culture surfaces can retain the ability to modulate their secretome in response to 30 kPa surfaces, MSCs from p3 can be seeded onto either TCP or 30 kPa RGD surfaces to assess changes in secretome, as further illustrated in FIG. 9a. MSCs serially passaged on 100 kPa RGD cell culture surfaces can retain the ability to increase secretion of many of the same growth factors, cytokines, and chemokines, as in FIG. 6a, when transferred to 30 kPa cell culture surfaces compared to TCP. The highest secreted factors, VEGF, IL-8 and GRO, are shown in FIG. 9c. These data suggest that the MSCs cultured on 100 kPa cell culture surfaces can retain the ability to discriminate between ultrastiff TCP and softer 30 kPa cell culture surfaces, similar to cells passaged directly from rescue culture (“p1”).

MSCs cultured on 30 kPa cell culture surfaces can broadly enhance the secretion of detected immunomodulatory and regenerative factors versus stiffer culture surfaces. MSCs cultured on 100 kPa cell culture surfaces can promote proliferation and increase secretion of selected factors compared to TCP. Extended culture on 100 kPa RGD cell culture surfaces can significantly reduce senescence-associated 0-galactosidase activity in MSCs versus TCP, while maintaining the capacity of the cells to enhance their secretome in response to 30 kPa versus TCP surfaces. Taken together, such embodiments identify cell culture surface culture parameters that can be utilized either to stimulate a heightened therapeutic secretome in cultured MSCs, or to expand healthy (non-senescent) MSCs that retain the ability to be primed.

Not only can certain cell culture surface culture surfaces improve the functional capacity of MSC secretome, but they can also improve MSC fitness as well. Culture on 100 kPa RGD cell culture surfaces, while maintaining similar levels of cell expansion to TCP, can reduce markers of replicative senescence seen during cell expansion, as shown in FIG. 9b. Such a decrease when cultured on cell culture surfaces softer than TCP suggests that MSCs may respond to TCP as a supra-physiologically stiff substrate, augmenting the development of senescence. Therefore, such examples can provide a strategy to reduce MSC replicative senescence using specialized culture substrates, offering a novel strategy to improve the production of clinical-scale quantities of non-senescent, fit MSCs.

While the present disclosure has been described in connection with a plurality of exemplary aspects, as illustrated in the various figures and discussed above, it is understood that other similar aspects can be used, or modifications and additions can be made to the described aspects for performing the same function of the present disclosure without deviating therefrom. For example, in various aspects of the disclosure, methods and compositions were described according to aspects of the presently disclosed subject matter. However, other equivalent methods or composition to these described aspects are also contemplated by the teachings herein. Therefore, the present disclosure should not be limited to any single aspect, but rather construed in breadth and scope in accordance with the appended claims.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the various patent offices and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. Instead, it is intended that the invention is defined by the claims appended hereto.

1. A tuneable cell culture surface comprising:
one or more acrylated hydrogel polymers; and
one or more functional compounds comprising one or more functional moieties;
wherein a mechanical property of the surface is tuneable based upon at least one of:
weight percent of one or more of the acrylated hydrogel polymers; and
molecular weight of one or more of the acrylated hydrogel polymers;
wherein the mechanical property is selected from the group consisting of stiffness, Young's modulus, elastic modulus, compressive modulus, and Poisson's ratio;
wherein a biochemical property of the surface is tuneable based upon selection of one or more of the functional compounds;
wherein the biochemical property is selected from the group consisting of cellular adhesion, cellular proliferation, cellular senescence, and cellular secretion; and
wherein the tuneable cell culture surface has a compressive modulus in the range from 10 kPa to 1000 kPa.

2. The tuneable cell culture surface of claim 1, wherein the tuneable cell culture surface comprises a tuneable solid hydrogel culture surface; and
wherein one or more of the functional moieties is selected from the group consisting of RGD moieties, HAVDI moieties, carbohydrate moieties, and lipid moieties.

3. The tuneable cell culture surface of claim 2, wherein the one or more functional compounds comprise at least one glycosaminoglycan (GAG) compound selected from the group consisting of: heparins, desulfated heparins, hyaluronic acids, and linear repeating disaccharides.

4. The tuneable cell culture surface of claim 1, wherein one of the functional compounds comprise a glycosaminoglycan (GAG) compound, a cadherin-engaging compound, an integrin-engaging compound, and a carbohydrate compound.

5.-6. (canceled)

7. A tuneable cell culture surface of a polymeric hydrogel comprising:
acrylated hydrogel polymers, at least one of which comprising poly(ethylene glycol) diacrylate (PEG-DA);
one or more functional moieties; and
optionally one or more cleavable crosslinkers;
wherein the one or more functional moieties include moieties that directly interact with cell surface receptors;
wherein one of the one or more functional moieties are selected from the group consisting of acrylated peptides RGD and HAVDI, glycosaminoglycan (GAG), methacrylated heparin and its derivatives, hyaluronic acid and its derivatives, and lipid moieties.
wherein the concentration of the one or more functional moieties ranges from 0 to 3 mM; and
wherein the weight percent of the acrylated hydrogel polymers is between 10% and 20%.

8. The tuneable cell culture surface of claim 7, wherein the acrylated hydrogel polymers have an average molecular weight from 600 Da to 10000 Da;
wherein the polymeric hydrogel comprises at least two functional moieties, GAGs and RGD;
wherein the GAGs are configured to localize cell-secreted factors at the tuneable cell culture surface in combination with the integrin-engaging RGD.

9. (canceled)

10. The cell culture surface of claim 1, further comprising an initial quantity of animal cells distributed on a surface of the cell culture surface.

11. The cell culture surface of claim 10, wherein the initial quantity of animal cells increases by a factor of from 1 to 10 after 4 days of culturing.

12. A process of culturing cells using the surface of claim 1 comprising:
distributing a culture of animal cells on the tuneable cell culture surface, wherein at least one moiety from the one or more functional moieties is configured to adhere to animal cells;
incubating the culture of animal cells such that the culture of animal cells attaches to the tuneable cell culture surface; and
recovering the culture of animal cells from the tuneable cell culture surface.

13. The method of claim 12, wherein the culture of animal cells comprises mesenchymal stem cells.

14. The method of claim 12, wherein the one or more functional compounds comprise at least one glycosaminoglycan (GAG) compound.

15. The method of claim 14, wherein the at least one GAG compound is selected from the group consisting of: heparins, desulfated heparins, hyaluronic acids, and linear repeating disaccharides.

16. The method of claim 12, wherein the one or more functional compounds comprise at least one cadherin-engaging compound.

17. The method of claim 12, wherein the one or more functional compounds comprise at least one integrin-engaging compound.

18. The method of claim 12, wherein the one or more functional compounds comprise at least one carbohydrate compound.

19. The method of claim 12, wherein one of the acrylated hydrogel polymers comprise poly(ethylene glycol) diacrylate (PEG-DA).

20. The method of claim 12, wherein the acrylated hydrogel polymers have an average molecular weight from 600 Da to 10000 Da.

21. The method of claim 12, wherein the acrylated hydrogel polymers are present in the tuneable cell culture surface in an amount from 1% to 75% by weight per unit volume.

22. The method of claim 12, wherein the culture of animal cells increases by a factor of from 1 to 10 after 4 days of culturing.

23. A method of making the tuneable cell culture surface of claim 1 comprising:
dissolving a polymer material comprising an acrylate functional group in a first solvent to create a first solution;
functionalizing the first solution with the one or more functional compounds; and
cross-linking the first solution to obtain the tuneable cell culture surface comprising the one or more acrylated hydrogel polymers.

24.-31. (canceled)
